(19) United States
(12) Patent Application Publication
(10) Pub. No.: US 2009/0035857 A1
(43) Pub. Date: Feb. 5, 2009

(54) CELL CULTURE PROCESSING DEVICES AND METHODS

(75) Inventors: Jonathan Chesnut, Carlsbad, CA (US); Robert Dees, San Diego, CA (US); Soojung Shin, Germantown, MD (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 12/180,473

(22) Filed: Jul. 25, 2008

Related U.S. Application Data
(60) Provisional application No. 60/953,896, filed on Aug. 3, 2007.

(51) Int. Cl. C12N 5/06; C12M 1/00

(52) U.S. Cl. 435/396; 435/298.2

(57) ABSTRACT

Embodiments are directed to devices and methods for processing, cultivating or otherwise manipulating cell cultures which may be disposed on a flat or substantially flat surface such as cell culture substrate material. Devices and methods are disclosed for dividing a cell culture layer into divided portions, including isolated divided portions, that may then be transferred from the cell culture to a new location. For some embodiments, the divided portions may be transferred to a new cell culture support substrate in order to continue to grow and cultivate the cell line.

### BACKGROUND

Cell culturing of specific cell lines or cells with specific attributes is an important aspect of many research and commercial production endeavors. Having a ready supply of uniform cells or cells with one or more desirable characteristics for research and product development is important to the advancement of work along these lines. Many types of cells, including human stem cells, can be seeded onto a surface of a growth substrate or other surface and proliferate into a colony of cells that forms a layer on the surface. These types of cells tend to thrive in an environment where they grow in close proximity to other cells of the same type. Once the cell culture has proliferated so as to produce a desired amount of cells, they may then be harvested. However, for cells such as human stem cells, as the cell colony proliferates and grows, the density of the cell population increases and the cells may begin to differentiate. In some cases, undifferentiated cells are desired for harvesting, so it may be important to remove the cells from the culture prior to differentiation or have the ability to segregate the differentiated cells from the undifferentiated cells prior to harvesting the cells.

Currently, the segregation of divided portions of cells from a layer of a cell colony growing on a substrate has been carried out by a painstaking process that requires a great deal of manual dexterity. In this process, a tool having a very fine tip point is made by hand by heating a portion of a glass pipette until the portion of the glass pipette is molten and malleable. The ends of the glass pipette are then pulled apart axially until the molten portion draws down to a fine thread about 20 to about 100 microns in diameter. The fine thread portion is then broken off at a position having a desired diameter and the original diameter portion of the pipette forms a handle portion. A user can then use the fine tip portion of the pipette tool as a tool to cut squares into or otherwise segment the layer of cell culture on the growth substrate by forming a criss-cross pattern by hand or other suitable method. Once the layer of cell culture has been cut into divided portions, the user may select divided portions of cells having desired attributes for selective removal of the divided portion from the layer of cell culture and relocation to a new growth substrate or other destination. The size of the cell culture layer may be several square inches and be divided into as many as several hundred divided portions or more. As such, this is a tedious and time consuming process that must be carried out under a microscope by a skilled operator.

What has been needed are devices and methods to allow a user to separate or partition a cell culture layer into divided portions having cells with one or more desirable attributes or a desired state without the need for handmade tools and tedious time intensive processes. What has also been needed are devices and methods for processing cell culture layers, generally, in an efficient and reliable manner.

### SUMMARY

Some embodiments of a cell culture processing device include a roller body having a substantially cylindrical outer surface with a layer penetrating structure 16 disposed thereon. The layer penetrating structure may be configured to penetrate a cell culture layer and partition the layer into divided portions. A support structure may be coupled to the roller body and configured to allow rotation of the roller body about an axis that is substantially concentric with the cylindrical outer surface. For some embodiments, the layer penetrating structure includes a plurality of adjacent circumferential ridges which may be regularly spaced in an axial direction. For some embodiments, the layer penetrating structure includes ridges surrounding substantially closed boundaries disposed at regularly space intervals on the cylindrical outer surface. For some embodiments, the outer surface of the roller body includes an elastomeric material.

Some embodiments of a cell culture processing tool include a roller body having a substantially cylindrical non-adherent outer surface with a layer penetrating structure configured to penetrate and partition a cell culture layer into divided portions disposed thereon. An axle may extend coaxially through the roller body and may be configured to support smooth rotational movement of the roller body about a longitudinal axis of the roller body. A handle may be coupled to the axle so as to allow rotational movement of the roller body about a longitudinal axis of the roller body relative to the handle. For some embodiments, the layer penetrating structure includes a plurality of adjacent circumferential ridges which may be regularly spaced in an axial direction. For some embodiments, the layer penetrating structure includes ridges surrounding substantially closed boundaries disposed at regularly spaced intervals on the cylindrical outer surface. For some embodiments, the handle includes an elongate handle body with a deflected distal section that forms an angle with a nominal longitudinal axis of the elongate handle body with the axle being coupled to the deflected distal section.

Some embodiments of a robotic cell culture processing tool may include a three axis robotic positioning actuator and a controller coupled to the three axis robotic positioning actuator. The processing tool may also include a roller body having a substantially cylindrical non-adherent outer surface with a layer penetrating structure configured to penetrate and partition a cell culture layer into divided portions. A support structure may be coupled to the roller body and configured to allow rotation of the roller body about an axis that is substantially concentric with the cylindrical outer surface. The support structure may also be secured to a movable carrier of the three axis robotic positioning actuator. For some embodiments, the layer penetrating structure includes a plurality of adjacent circumferential ridges which are regularly spaced in an axial direction. For some embodiments, the layer penetrating structure includes ridges surrounding substantially closed boundaries disposed at regularly spaced intervals on the cylindrical outer surface. For some embodiments, the controller includes a processor which is programmed to controllably move the roller body in a pre-determined pattern of motion which may be at least one linear pass across a cell culture layer disposed in a cell culture dish.

Some embodiments of a method of processing a cell culture layer include separating the cell culture layer by advancing a roller body having a layer penetrating raised structure across the cell culture layer. The roller body may be advanced across the cell culture layer while applying a predetermined amount of force against the cell culture layer to cut through the cell culture layer and partition the cell culture layer into divided portions. For some embodiments, the layer penetrating structure includes ridges surrounding substantially closed boundaries disposed at regularly spaced intervals on the cylindrical outer surface of the roller body and a single pass of the roller body across the cell culture layer is used to separate cell culture layer into isolated divided portions. For some embodiments, the layer penetrating structure of the roller body includes circumferential ridges spaced axially from each other and at least two passes of roller body across the cell culture layer in at least two different directions are used to separate cell culture layer into isolated divided portions.

Some embodiments of a method of processing a cell culture include seeding a cell culture support substrate with a cells and allowing the cells of the cell line to proliferate on the cell culture substrate and form a cell culture layer disposed on the cell culture substrate surface. The cell culture layer may then partitioned by advancing a roller body having a layer penetrating raised structure across the cell culture layer while applying a predetermined amount of force against the cell culture layer to cut through the cell culture layer and partition the cell culture layer into divided portions.

Some embodiments of a method for passaging cells may include partitioning a cell culture layer with a cell culture processing tool by rolling a layer penetrating structure of the cell culture processing tool over the cell culture layer so as to partition the layer of cells into a plurality of isolated divided portions. An isolated divided portion of the layer of cells may be selected having cells with at least one predetermined characteristic. The isolated divided portion may then be transported to a new location the transported cells allowed to proliferate in the new location and generate a new cell culture layer of cells having the predetermined characteristic. For some embodiments, partitioning the cell culture layer may include partitioning the cell culture layer into isolated divided portions with a single pass of a roller body having a layer penetrating structure which comprises ridges surrounding substantially closed boundaries disposed at regularly space intervals on a cylindrical outer surface of the roller body. For some embodiments, the layer penetrating structure may include a plurality of adjacent circumferential ridges regularly spaced in an axial direction on a cylindrical outer surface of the roller body and partitioning the cell culture layer may include partitioning the cell culture layer into isolated divided portions with two passes of the roller body across the cell culture layer in different directions. Some embodiments may include repeating this passaging method for about 1 passage to about 1000 passages.

Some embodiments for a method of maintaining a cell line in a desired state may include partitioning a cell culture layer with a cell culture processing tool by rolling a layer penetrating structure of the cell culture processing tool over the cell culture layer so as to partition the layer of cells into a plurality of isolated divided portions. An isolated divided portion of the layer of cells may be selected having cells in a predetermined state. The isolated divided portion may then be transported to a new location and the transported cells allowed to proliferate in the new location and generate a new cell culture layer of cells having the predetermined state. For some embodiments, partitioning the cell culture layer may include partitioning the cell culture layer into isolated divided portions with a single pass of a roller body having a layer penetrating structure which comprises ridges surrounding substantially closed boundaries disposed at regularly space intervals on a cylindrical outer surface of the roller body. For some embodiments, the layer penetrating structure may include a plurality of adjacent circumferential ridges regularly spaced in an axial direction on a cylindrical outer surface of the roller body and partitioning the cell culture layer may include partitioning the cell culture layer into isolated divided portions with two passes of the roller body across the cell culture layer in different directions.

These features of embodiments will become more apparent from the following detailed description when taken in conjunction with the accompanying exemplary drawings.

### BRIEF DESCRIPTION OF THE DRAWINGS

FIG. 1 is a perspective view of an embodiment of a cell culture processing tool.

FIG. 2 is an elevation view of the cell culture processing tool of FIG. 1.

FIG. 3 is a perspective view of an embodiment of a roller body of the cell culture processing tool of FIG. 1.

FIG. 4 is an elevation view of the roller body of FIG. 3.

FIG. 5 is an enlarged view of the roller body surface of FIG. 3

FIG. 6 is an elevation view in partial section of the roller body of FIG. 3.

FIG. 7 is an enlarged view of the roller body in section.

FIG. 8 is a perspective view of an embodiment of a roller body of a cell culture processing tool.

FIG. 9 is a perspective view of an embodiment of a roller body of a cell culture processing tool.

FIG. 10 is an elevation view of the roller body of FIG. 9.

FIG. 11 is an enlarged view of a surface of the roller body of FIG. 9.

FIG. 11A shows a sectional view of partitioning of a cell culture layer by circumferential ridges of a layer penetrating structure of the roller body.

FIG. 12 is a perspective view of a cell culture dish having a layer of cell culture layer disposed on a layer of cell culture support substrate.

FIG. 13 is an elevation view of the cell culture dish of FIG. 12 in section.

FIG. 14 is an enlarged view of the cell culture layer and adjacent layer of cell culture support substrate.

FIG. 15 is a top view of a cell culture dish with a cell culture sample being deposited on a top surface of cell culture support substrate.

FIG. 16 is a top view of the cell culture dish of FIG. 15 with a cell culture layer growing on the surface of the cell culture layer support substrate.

FIG. 17 is a top view of the cell culture dish of FIG. 16 showing a roller body of a cell culture processing tool advancing across the cell culture layer.

FIG. 17A is an enlarged sectional view of outer edges of circumferential ridges of a layer penetrating structure on the surface of a roller body penetrating and partitioning a cell culture layer disposed on a support layer as the roller body is advanced across the cell culture layer.

FIG. 18 is a top view of the cell culture dish of FIG. 17 with substantially parallel linear cuts in the cell culture layer.

FIG. 19 is a top view of the cell culture dish of FIG. 18 with the cell culture processing tool advancing across the cell culture layer in a substantially perpendicular orientation relative to the linear cuts shown in FIG. 18.

FIG. 20 is a top view of the cell culture dish of FIG. 19 with the cell culture layer divided into substantially square divided portions.

FIG. 21 is an enlarged view of the cell culture layer of FIG. 20 showing the divided portions of the cell culture layer in more detail.

FIG. 22 is a top view of the cell culture dish of FIG. 20 with a divided portion of the cell culture layer being lifted and removed.

FIG. 23 is a top view of a cell culture layer of human stem cells showing differentiated cell portions and undifferentiated cell portions.

FIG. 24 is a top view of a cell culture layer of human stem cells which has been divided into substantially square isolated divided portions with a cell culture processing tool.

FIG. 25 is a side view of an embodiment of a robotic cell culture processing tool.

FIG. 26 is a front elevation view of the robotic cell culture processing tool of FIG. 25.

### DETAILED DESCRIPTION

Embodiments of the invention relate generally to the cultivation of cells. In particular embodiments, the invention relates to devices, compositions and methods for cell cultivation, including prolonged cell cultivation. In some embodiments, the invention relates to the maintenance of cells in a particular state over an extended period of time. For example, embodiments may be used to maintain cells in a state of non-differentiation or differentiation, over an extended period of time of about 1 day to about 5 years, more specifically, about 3 days to about 1 year, and even more specifically, about 5 days to about 1 month. For some embodiments, cells may be maintained in a desired state for about 1 passage to about 1000 passages, more specifically, about 5 passages to about 500 passages, and even more specifically, about 20 passages to about 100 passages.

Embodiments of cell culture processing tools discussed herein have the ability to partition a cell culture layer into divided portions, including isolated divided portions, each having substantially the same surface area without cross contaminating the cells of one divided portion with the cells of another divided portion. Cell culture processing tool embodiments discussed herein may cut or otherwise create a partition through a cell culture layer without picking up cells as a layer penetrating structure of a roller body of the tool advances across the cell culture layer. As such, it may be desirable for an outer surface of the roller body to have a substantially non-adherent surface that may also be sterile and inert to avoid contamination of the cells of the cell culture layer with contaminants that are either disposed on the roller body or emanate from the material of the roller body. Divided portions of the cell culture layer may need to be isolated from all adjacent portions of the cell culture layer and remain in contact with an optional support substrate after partitioning to allow the divided portions to be lifted or otherwise removed from the support substrate. As such, it may be desirable in some embodiments for the roller body and layer penetrating structure thereof to be configured to prevent skidding or sliding of the layer penetrating structure as it is advanced across a cell culture layer. Embodiments of the cell culture processing tool may be economical to manufacture and configured as single use devices.

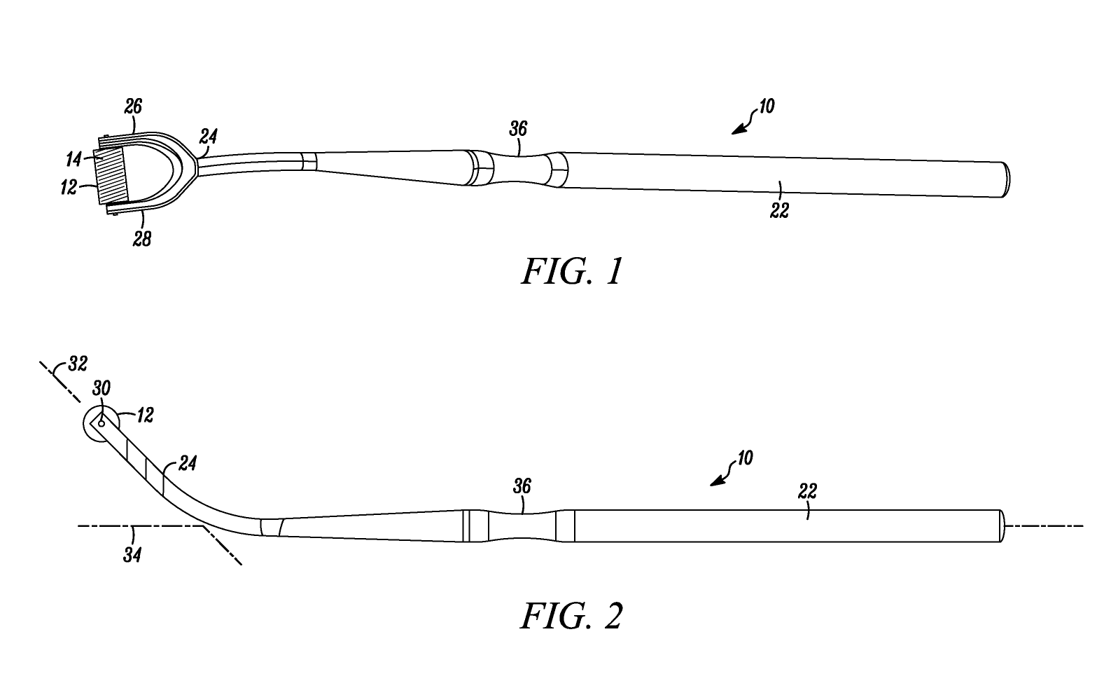

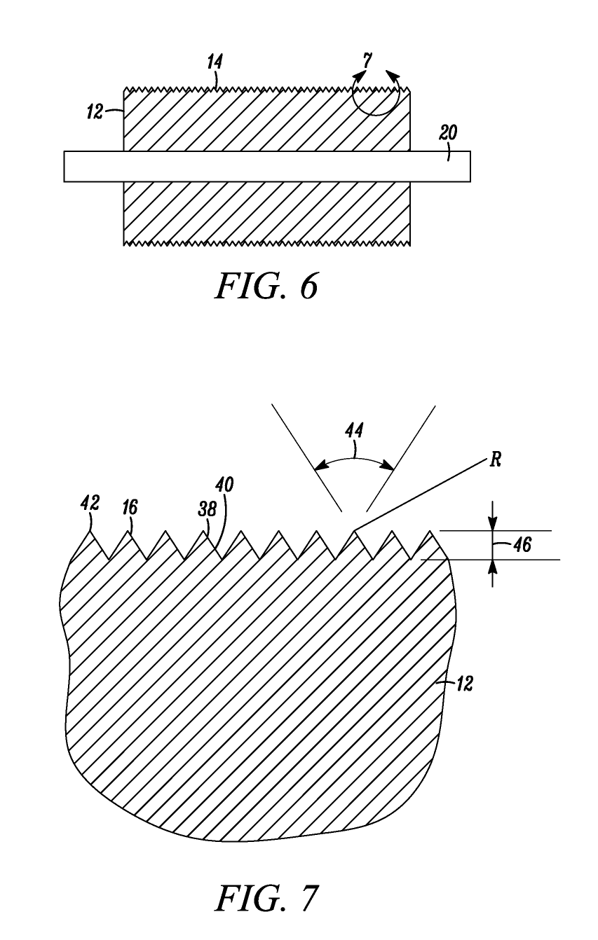

FIGS. 1-7 illustrate an embodiment of a cell culture processing tool 10 that may be used to separate or partition a cell culture layer disposed on a flat or substantially flat support, which may optionally include a support substrate layer disposed in a cell culture dish, into divided portions. The divided portions produced by the cell culture processing tool 10 may be selectively removed from the support layer below the cell culture layer and transferred from the cell culture dish to another location, such as another cell culture dish, test tube or any other desired location. In this way, cells of the cell culture having desired characteristics may be further grown and cultured in order to produce more of the same in a reliable and repeatable manner.

The cell culture processing tool 10 includes a roller body 12 having a cylindrical non-adherent outer surface 14 with a layer penetrating structure 16 disposed on the outer surface 14 of the roller body 12. The layer penetrating structure 16 is configured to penetrate a cell culture layer and partition the cell culture layer into divided portions which may assume a variety of shapes and sizes. The roller body 12 in FIG. 1 is configured to rotate about a longitudinal axis 18 of the roller body 12 which is substantially concentric with the outer surface 14 of the roller body 12. This rotation may be supported by a support structure that includes an axle 20 that extends coaxially through the roller body 12 and which is configured to support smooth rotational movement of the roller body 12 about the longitudinal axis 18 of the roller body 12. The support structure may also include a handle 22 which is coupled to the axle 20 so as to allow rotation of the axle 20 and roller body 12. A deflected distal section 24 of the handle 22 includes a bifurcated structure having a first leg 26 and a second leg 28 with distal portions of each leg of the bifurcated structure having opposed recesses 30 that capture respective ends of the axle 20 and allow rotation of the axle 20 within the recesses 30. For the embodiment shown, the longitudinal axis 18 of the roller body 12 and axle 20 are substantially perpendicular to a plane defined by a longitudinal axis 32 of the deflected distal section 24 and a longitudinal axis 34 of the elongate handle body proximal of the deflected distal section 24.

Referring to FIGS. 1 and 2, the handle body proximal of the deflected distal section 24 of the embodiment shown has an ergonomic design that allows a user to grasp the handle 22 manually and controllably manipulate the roller body 12 at the distal end of the handle 22. The deflected distal section 24 may allow a user to position the roller body 12 on a cell culture layer disposed within a circular cell culture dish with the roller body 12 disposed against an inside wall of a culture dish. The user may control movement of the roller body across a surface of a cell culture layer disposed on a support substrate within the cell culture dish by grasping the ergonomic handle 22. The deflected distal section 24 may be configured to allow the roller body 12 to be brought into contact with the side wall of the culture dish while still holding the handle 22 in a comfortable position in a user's hand.

The handle 22 has a generally elongate cylindrical shape with a round transverse cross section and a waist portion 36 tapering to a reduced transverse diameter at about the middle point of the handle body between the proximal end of the handle and the distal ends of the legs 26 and 28 of the deflected distal section 24. The waist portion 36 provides a recess for an operator to grasp between thumb and forefinger to maintain control of the movement of the tool 10 in an axial direction while using the tool 10. Embodiments of the handle 22 may have a length of about 2 inches to about 10 inches, more specifically, about 3 inches to about 8 inches, and even more specifically, about 4 to about 7 inches, and a nominal transverse diameter or dimension of about 0.1 inches to about 0.7 inches, more specifically, about 0.2 inches to about 0.5 inches in the portion of the handle body proximal of the distal deflected section 24. The first and second legs 26 and 28 of the bifurcation in the deflected distal section 24 may have a length of about 0.2 inches to about 1.5 inches, more specifically, about 0.5 inches to about 1.0 inch. The length of the deflected distal section 24 may have a length of about 0.5 inch to about 2.5 inches, more specifically, about 1.0 inch to about 1.5 inches, and may form an angle of about 110 degrees to about 160 degrees, more specifically, about 120 degrees to about 150 degrees with respect to the nominal longitudinal axis 34 of the elongate handle body 22.

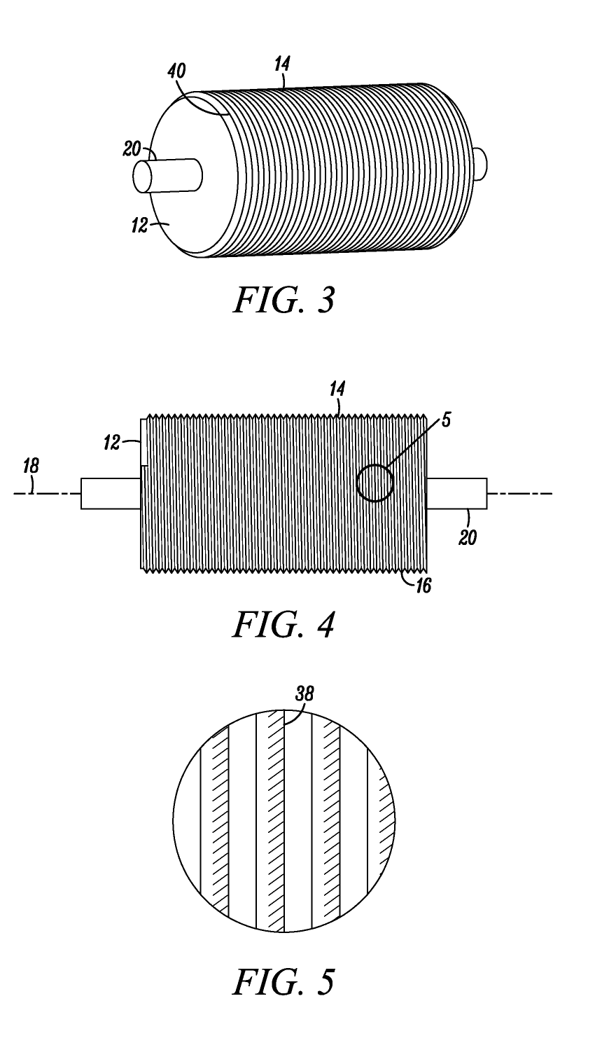

Referring to FIGS. 3-7, the roller body 12 may have a generally cylindrical outer surface 14 that includes layer penetrating structure 16. It may be desirable for the outer surface 14 to be a non-adherent outer surface 14 so that when the roller body 12 is advancing across a cell culture layer, the cells of one portion of the cell culture layer are not picked up and transported to another portion of the cell culture layer which might result in cross contamination. It may also be desirable for the outer surface 14 to be sterile and inert so as to avoid contamination of the cells with foreign contaminants. The layer penetrating structure 16 for the embodiment shown includes a plurality of adjacent circumferential ridges 38 regularly spaced in an axial direction on the cylindrical outer surface 14 of the roller body 12. For the embodiment shown, the plurality of adjacent circumferential ridges 38 are formed from a single helical ridge 40 which extends from a first axial end of the roller body 12 to a second axial end of the roller body 12 which is axially spaced from the first axial end. The roller body embodiment 12 shown may be formed by a variety of processes, such as molding, machining, etching or the like from any suitable material that will allow for a non-adherent outer surface with suitable mechanical properties. For some embodiments, suitable materials for the roller body may include elastomers such as silicone rubber, polyurethanes, polytetrafluoroethylenes, nylons, stainless steel and the like. For some embodiments, the roller body 12 may be formed by molding an elastomer material into a solid structure having a desired configuration of that provides a firm but somewhat compliant structure with a non-adherent outer surface 14. In addition, although the embodiment of the roller body 12 is shown as a solid structure, it may also be desirable to form the roller body 12 to include interior voids, slots, grooves, spokes or other structures in order to save weight and material.

The elastomer material of the roller body 12 is molded over the elongate cylindrical axle 20 in a monolithic structure which is secured in fixed concentric relation to the roller body 12. The elastomer provides a material that is temperature stable, moldable, inert and non-adherent. The elastomer may also be sterilized by methods such as gamma irradiation or other suitable methods. Suitable materials for the roller body 12, outer surface 14 of the roller body 12 and layer penetrating structure 16 of the roller body 12 may include elastomers such as silicone rubber, polyturethanes as well as other suitable materials that provide the above characteristics. The first end and second end of the axle 20 are sized to freely rotate within the recesses 30 at the distal ends of the respective legs 26 and 28 of the bifurcated structure of the handle 22. For such embodiments, the recesses 30 in the ends of the legs 26 and 28 of the bifurcated structure of the handle 22 may be cylindrical concentric holes that extend through the distal portions of the legs 26 and 28. The holes may be through holes or blind holes that open to an interior portion between the two legs 26 and 28 of the bifurcated structure. In order for the roller body 12 to rotate freely about the axle 20 during use in partitioning a cell culture layer, it has been found that for some embodiments, it is useful for the portions of the axle 20 that rotate within the recesses 30 to have a small outer diameter relative the outer diameter of the roller body 12.

This structure provides free rotational motion of the roller body 12 to prevent skidding, sliding or plowing of the layer penetrating structure 16 of the roller body 12 over a cell culture layer as the roller body 12 is being rotated and advanced across the cell culture layer. Sliding, skidding or plowing of the layer penetrating structure 16 across the cell culture layer may produce cross contamination or deformation of the cell culture layer as well as other undesirable effects. For such embodiments, the shear resistance or resistance to inelastic shear deformation of the cell culture layer in contact with roller body 12 should be greater than the frictional resistance to rotation of the roller body 12 at the outer surface 14 of the roller body 12. In addition, the frictional force in shear between the outer surface 14 of the roller body 12 and the cell culture layer must be greater than the resistance to rolling of the roller body 12 at the outer surface 14 thereof. For some embodiments, the axle 20 may have an outer diameter that is about 10 percent to about 25 percent of the outer diameter of the roller body 12, more specifically, about 12 percent to about 22 percent, and even more specifically, about 15 percent to about 20 percent. For some embodiments, the axle 20 may have an outer diameter of about 0.02 inch to about 0.08 inch, more specifically, about 0.03 inch to about 0.05 inch and may be made from a suitable inert high strength material such as stainless steel or the like.

For some embodiments, the material of the roller body 12 and of the layer penetrating structure 16 may have a shore hardness of about 60 A to about 80 A, more specifically, about 65 A to about 75 A, and even more specifically, about 68 A to about 72 A. For some embodiments, the circumferential ridges 38 may be about 0.003 inches high to about 0.015 inches high, more specifically, about 0.005 inches high to about 0.012 inches high, and even more specifically, about 0.007 inches high to about 0.010 inches high, as indicated by arrow 46 in FIG. 7. For some embodiments, the circumferential ridges 38 may be about 0.015 inches high to about 0.06 inches high, more specifically, about 0.02 inches high to about 0.05 inches high, and even more specifically, about 0.025 inches high to about 0.04 inches high. For some embodiments, the circumferential ridges 38 may be spaced axially apart by a length of about 0.003 inches to about 0.015 inches, more specifically, about 0.005 inches to about 0.012 inches, and even more specifically, about 0.007 inches to about 0.010 inches apart for some embodiments. The circumferential ridges 38 may also be spaced axially apart by a length of about 0.015 inches to about 0.06 inches, more specifically, about 0.02 inches to about 0.05 inches, and even more specifically, about 0.025 inches to about 0.04 inches apart for some embodiments. For some embodiments, the circumferential ridges 38 may have an angle, as indicated by arrow 44 in FIG. 7, of about 40 degrees to about 80 degrees, more specifically, about 50 degrees to about 70 degrees, and even more specifically, about 55 degrees to about 65 degrees.

The partitioning outer edge 42 of the ridge 40, or individual axially adjacent circumferential ridges 38, may have a radius of curvature R as indicated in FIG. 7. Some embodiments of the ridges 38 of the roller body 12 may have a radius of curvature of about 0.0002 inch to about 0.001 inch, more specifically, about 0.0003 inch to about 0.0009 inch. The radius of curvature R of the partitioning outer edge 42 of the ridge 40 may be critical in some embodiments in order to provide a partitioning outer edge 42 that is sharp enough to reliably cut through and partition a cell culture layer while remaining stable and not rolling over, folding or otherwise deforming in such a way during the partitioning process so as to detract from the efficiency of the tool 10. The ridge angle 44 may also be important in this regard for some embodiments.

Embodiments of the roller body 12, and layer penetrating structure 16 thereof, may have an axial length of about 0.2 inch to about 1 inch, more specifically, about 0.3 inch to about 0.5 inch, and even more specifically, about 0.35 inch to about 0.45 inch. The roller body 12 and layer penetrating structure thereof may have an outer diameter of about 0.05 inch to about 1 inch, more specifically, about 0.1 inch to about 0.5 inch, and even more specifically, about 0.15 inch to about 0.25 inch, for some embodiments. Embodiments of the roller body 12, and layer penetrating structure 16 thereof, may have an axial length of about 1 inch to about 3 inches, more specifically, about 1.5 inches to about 2.5 inches, and even more specifically, about 1.8 inches to about 2.2 inches. The roller body 12 and layer penetrating structure thereof may have an outer diameter of about 0.5 inch to about 2 inches, more specifically, about 0.6 inch to about 1.5 inch, and even more specifically, about 0.8 inch to about 1.2 inch, for some embodiments.

The ridges 38 of the layer penetrating structure 16, as shown in more detail in FIGS. 5 and 7, extend in a circumferential direction substantially parallel to each other and have a cutting or partitioning outer edge 42 that is configured to penetrate and partition a cell culture layer. As discussed above, the circumferential ridges 38 are formed from the single helical ridge 40 that extends from the first end of the roller body 12 to the second end of the roller body 12. This structure allows the roller body 12 to be conveniently manufactured by making a two piece mold (not shown) that may have an interior cavity surface threads formed thereon by a thread cutting tool. The threaded interior surface of such a mold may be configured to have a mirror image of the dimensions and configurations of any of the embodiments of the ridges 38 discussed above. The mold may then be used to form a layer penetrating structure 16 having a ridge 38 which has been molded by injecting a flowable material such as the elastomer or other suitable materials of the roller body 12 into and against the threaded interior surface.

For some embodiments, the threads cut into the interior surface of the mold may have a pitch in an axial orientation of about 50 threads per inch to about 150 threads per inch, more specifically, about 100 threads per inch to about 120 threads per inch, and even more specifically, about 105 threads per inch to about 110 threads per inch. The threads on the mold surface may also have a thread angle of about 40 degrees to about 80 degrees, more specifically, about 50 degrees to about 70 degrees, and even more specifically, about 55 degrees to about 65 degrees. The threads may have a thread height of about 0.005 inch to about 0.015 inch, more specifically, about 0.008 inch to about 0.012 inch.

Once the mold is formed, the elastomer of the roller body 12 may be injection molded into the mold over the axle 20 so as to economically form a high precision repeatable structure. The handle 22 may also be produced by injection molding of such materials as ABS plastic, polyurethane, or the like having a shore hardness of about 85 B to about 95 B, more specifically, about 86 B to about 94 B, and even more specifically, about 87 B to about 93 B, for some embodiments. Some flexibility of the handle during use may be beneficial for some embodiments. Such a structure may be produced inexpensively enough to allow the cell processing tool to be used as a single use or disposable device.

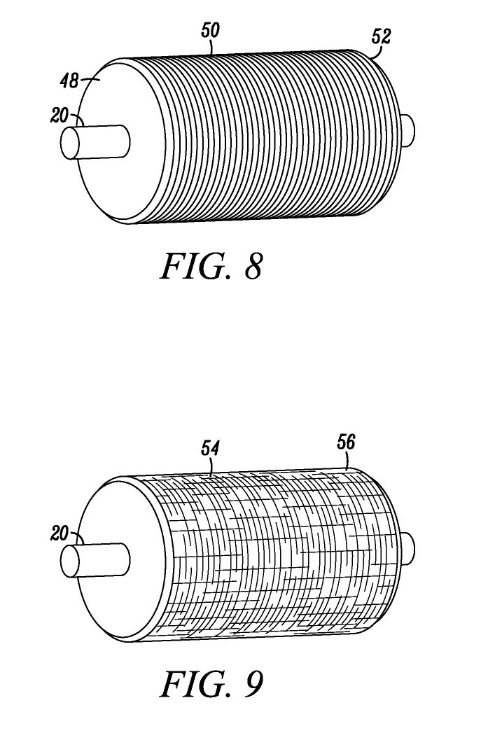

FIG. 8 illustrates an embodiment of a roller body 48 that may have features, dimensions and materials which are the same as or similar to the features, dimensions and materials of roller body 12 shown in FIGS. 1-7. However, for the embodiment 48 of FIG. 8, the layer penetrating structure 50 includes a plurality of circumferential ridges 52 which are configured as separate circumferential rings which are parallel and adjacent each other, but formed separately and not from a single helical ridge, such as helical ridge 40 discussed above, extending from one end of the roller body 48 to a second end of the roller body 48. Such a configuration is capable of producing a plurality of parallel and linear partition strips with a single pass which may extend over a long distance over many multiple rotations of the roller body 48. The ridges 52 of the layer penetrating structure 50 of this embodiment may be made from the same materials and have a range of angles, axial separation, height as well as other parameters as the ridges 38 of the roller body 12 in FIGS. 1-7.

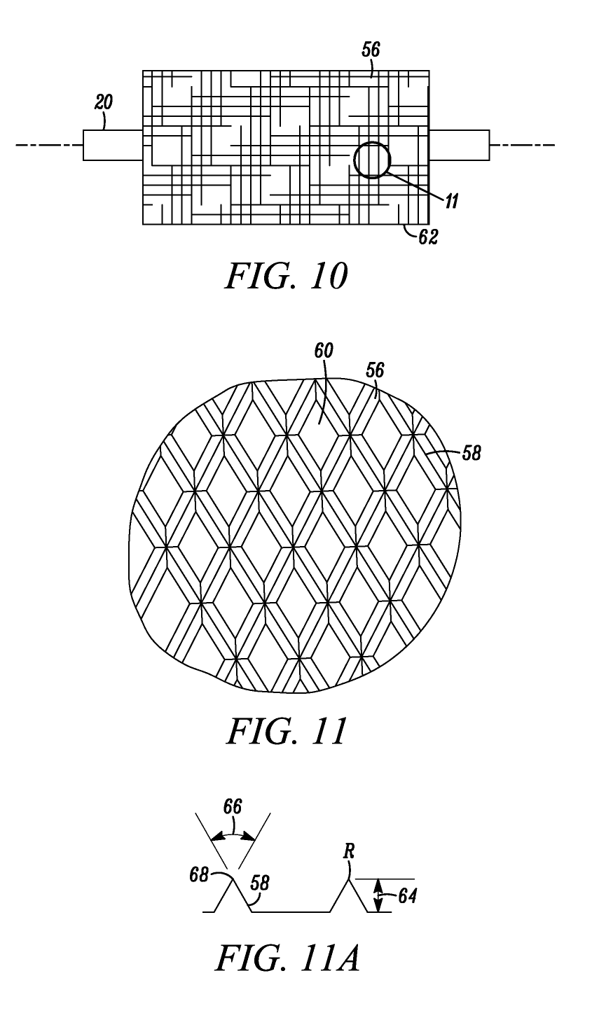

FIGS. 9-11A illustrate an embodiment of a roller body 54 having a layer penetrating structure 56 that includes ridges 58 surrounding substantially closed boundaries 60 disposed at regularly spaced intervals on the cylindrical outer surface 62 of the roller body 54. For such an embodiment, a single pass of the roller body 54 across a cell culture layer is capable of partitioning a cell culture layer into isolated divided portions in a single pass of the roller body across the cell culture layer, which may save time and expense during the partitioning process. For example, the roller body 12 embodiments illustrated in FIGS. 1-8 will generally require at least two passes of the roller body 12 across a cell culture layer in different directions in order to partition a cell culture layer into isolated divided portions of the cell culture layer as a single pass produces elongated strip divided portions that may or may not be isolated from adjacent strip divided portions as will be discussed in more detail below.

For the embodiment shown in FIGS. 9-11A, the substantially closed boundaries 60 are rectangular or diamond shaped. The materials, dimensions and features of the roller body 54 of FIGS. 9-11 may be the same as or similar to the features, dimensions and materials of the roller body 12 shown in FIGS. 1-7. The layer penetrating structure 56 is disposed on a cylindrically shaped outer surface 62 of the roller body 54 with the repeating pattern of closed boundaries 60 as shown. Each ridge of the enclosed boundary region 60 is configured to cut through a cell culture layer and partition the cell culture layer an isolated divided portions as the roller body 54 is advanced across the cell culture layer. The configuration of the individual ridge portions 58 of the layer 56 penetrating structure may include the same general materials, dimensions, features and configuration as those of the ridges 38 of the roller body 12 discussed above.

In particular, the ridge structures 58 surrounding the enclosed boundary portions 60 may be about 0.003 inches to about 0.015 inches high, more specifically, about 0.005 inches to about 0.012 inches high, and even more specifically, about 0.007 inches high to about 0.01 inches high, as shown by arrow 64 in FIG. 11A. The ridge structures 58 may also be spaced apart from each other about 0.003 inches to about 0.015 inches, more specifically, about 0.005 inches to about 0.012 inches, and even more specifically, about 0.007 inches apart to about 0.01 inches apart, at the point of widest separation across each enclosed boundary portion. The ridges 58 of the layer penetrating structure 56 may have an outward taper angle of about 40 degrees to about 80 degrees, more specifically, about 50 degrees to about 70 degrees, and even more specifically, about 55 degrees to about 65 degrees, as indicated by arrow 66 in FIG. 11A. The partitioning outer edge 68 of each ridge segment may have a radius of curvature R of about 0.0002 inch to about 0.001 inch, more specifically, about 0.0003 inch to about 0.0009 inch. In general, the ridge structures 58 may also have features, materials and dimensions which are the same as or similar to those of the circumferential ridge embodiments 38 discussed above.

The roller body 54, and layer penetrating structure 56 thereof, may have an axial length of about 0.2 inch to about 1 inch, more specifically, about 0.3 inch to about 0.5 inch, and even more specifically, about 0.35 inch to about 0.45 inch for some embodiments. The roller body 54 and layer penetrating structure 56 thereof may have an outer diameter of about 0.05 inch to about 1 inch, more specifically, about 0.1 inch to about 0.5 inch, and even more specifically, about 0.15 inch to about 0.25 inch, for some embodiments. For some embodiments, the material of the layer penetrating structure 56, which may include an elastomer material such as silicone rubber, as well as other suitable materials, may have a shore hardness of about 60 A to about 80 A, more specifically, about 65 A to about 75 A, and even more specifically, about 68 A to about 72 A. In general, the roller body 54 may have features, materials and dimensions which are the same as or similar to those of roller body 12 discussed above.

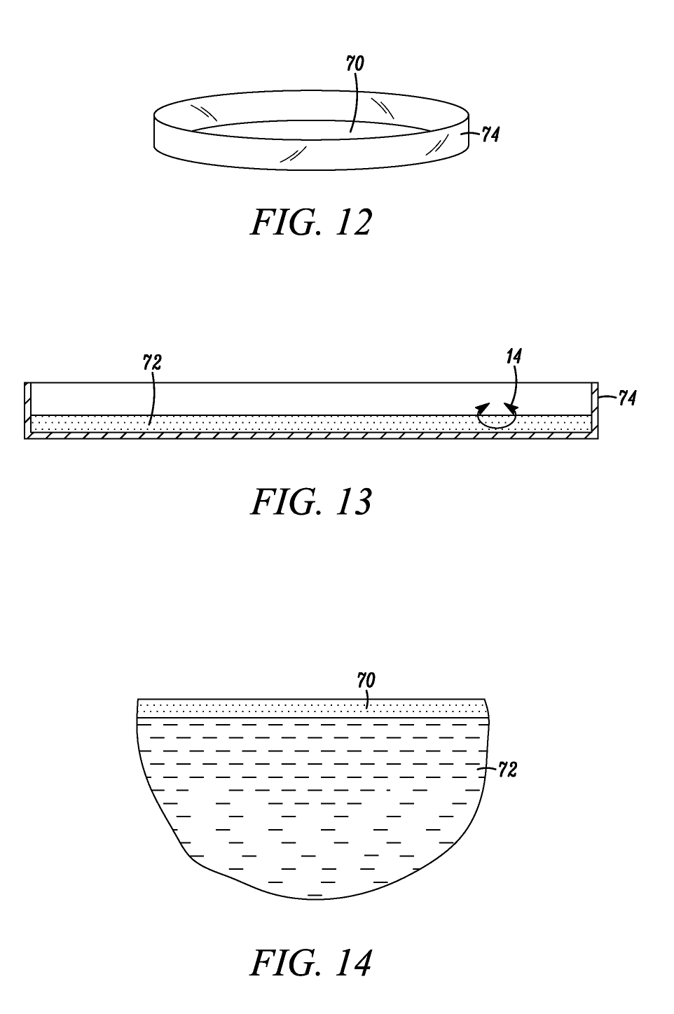

FIGS. 12-14 illustrate a cell culture layer 70 disposed on an optional support layer or substrate 72 in a cell culture dish 74 that may be used in conjunction with embodiments of the devices and methods described herein. The cell culture layer 70 may include a variety of cell cultures such as stem cells, including human stem cells, embryoid bodies, neurospheres and the like. The support layer 74 may include inactive support media such as agar, algae, mouse or human fibroblast feeder cells as well as other suitable materials. The support layer 72 generally provides a source of nutrients and physical support for the cell culture layer to proliferate. The support layer may generally have a depth or thickness of about 0.0001 mm to about 0.1 mm, more specifically, about 0.0002 mm to about 0.01 mm for some embodiments, and even more specifically, about 0.0005 mm to about 0.005 mm. Embodiments of the support layer are typically somewhat soft and pliable with a gel-like quality. It should also be noted that some cell culture layer embodiments 70 may also be grown directly on surfaces such as bottom surface of a cell culture dish 74 without the optional support layer 72. The cell culture layer 70 may be confluent or continuous, near confluent, semi-confluent or not confluent. Any combination of the cells or suitable cell culture support materials described herein may be used in conjunction with embodiments of the devices and methods described herein.

In addition to the cell culture layer cells and support layer materials discussed above, other cells and support layer materials may also be useful. Examples of animal cell culture media that may be prepared and used with embodiments of the present invention include without limitation DMEM, RPMI-1640, MCDB 131, MCDB 153, MDEM, IMDM, MEM, M199, McCoy's 5A, Williams' Media E, Leibovitz's L-15 Medium, Grace's Insect Medium, IPL-41 Insect Medium, TC-100 Insect Medium, Schneider's Drosophila Medium, Wolf & Quimby's Amphibian Culture Medium, cell-specific serum-free media (SFM) such as those designed to support the culture of keratinocytes, endothelial cells, hepatocytes, melanocytes, etc., F10 Nutrient Mixture and F12 Nutrient Mixture. Other media, media supplements and media subgroups suitable for preparation by the invention are available commercially (e.g., from Invitrogen, Inc.; Rockville, Md., and Sigma; St. Louis, Mo.). Formulations for these media, media supplements and media subgroups, as well as many other commonly used animal cell culture media, media supplements and media subgroups are well-known in the art and may be found, for example in the GEBCO/BRL Catalogue and Reference Guide (Life Technologies, Inc.; Rockville, Md.) and in the Sigma Animal Cell Catalogue (Sigma; St. Louis, Mo.).

Cells may be cultured in undefined or defined media in certain embodiments. To overcome drawbacks of the use of serum or organ/gland extracts, a number of so-called “defined” media have been developed. These media, which often are specifically formulated to support the culture of a single cell type, contain no undefined supplements and instead incorporate defined quantities of purified growth factors, proteins, lipoproteins and other substances usually provided by the serum or extract supplement. Since the components (and concentrations thereof) in such culture media are precisely known, these media are generally referred to as “defined culture media.” Often used interchangeably with “defined culture media” is the term “serum-free media” or “SFM.” A number of SFM formulations are commercially available, such as those designed to support the culture of endothelial cells, keratinocytes, monocytes/macrophages, fibroblasts, chondrocytes or hepatocytes which are available from GIBCO/LTI (Gaithersburg, Md.). The distinction between SFM and defined media, however, is that SFM are media devoid of serum, but not necessarily of other undefined components such as organ/gland extracts. Indeed, several SFM that have been reported or that are available commercially contain such undefined components, including several formulations supporting in vitro culture of keratinocytes (Boyce, S. T., and Ham, R. G., J. Invest. Dermatol. 81:33 (1983); Wille, J. J., et al., J. Cell. Physiol. 121:31 (1984); Pittelkow, M. R., and Scott, R. E., Mayo Clin. Proc. 61:771 (1986); Pirisi, L., et al., J. Virol. 61:1061 (1987); Shipley, G. D., and Pittelkow, M. R., Arch. Dermatol. 123:1541 (1987); Shipley, G. D., et al., J. Cell. Physiol. 138:511-518 (1989); Daley, J. P., et al., FOCUS (GIBCO/LTI) 12:68 (1990); U.S. Pat. Nos. 4,673,649 and 4,940,666). SFM thus cannot be considered to be defined media in the true definition of the term.

Defined media can provide several advantages to the user. For example, the use of defined media facilitates the investigation of the effects of a specific growth factor or other medium component on cellular physiology, which may be masked when the cells are cultivated in serum- or extract-containing media. In addition, defined media typically contain much lower quantities of protein (indeed, defined media are often termed “low protein media”) than those containing serum or extracts, rendering purification of biological substances produced by cells cultured in defined media far simpler and more cost-effective. Some extremely simple defined media, which consist essentially of vitamins, amino acids, organic and inorganic salts and buffers have been used for cell culture. Such media (often called “basal media”), however, are usually seriously deficient in the nutritional content required by most animal cells. Accordingly, most defined media incorporate into the basal media additional components to make the media more nutritionally complex, but to maintain the serum-free and low protein content of the media. Examples of such components include serum albumin from bovine (BSA) or human (HSA); certain growth factors derived from natural (animal) or recombinant sources such as EGF or FGF; lipids such as fatty acids, sterols and phospholipids; lipid derivatives and complexes such as phosphoethanolamine, ethanolamine and lipoproteins; protein and steroid hormones such as insulin, hydrocortisone and progesterone; nucleotide precursors; and certain trace elements (reviewed by Waymouth, C., in: Cell Culture Methods for Molecular and Cell Biology, Vol. 1: Methods for Preparation of Media, Supplements, and Substrata for Serum-Free Animal Cell Culture, Barnes, D. W., et al., eds., New York: Alan R. Liss, Sic., pp. 23-68 (1984), and by Gospodarowicz, D., Id., at pp 69-86 (1984); see also US).

Any type of cell that can be cultured may be utilized in conjunction with embodiments of the invention described herein. Animal cells that can be used include, but are not limited to, cells obtained from mammals, birds (avian), insects or fish. Cell types or cells in desired states, such as differentiated states, that can be utilized include without limitation embryonic cells, stem cells, fetal cells and differentiated cells (e.g., from brain, eye, skin (e.g., dermal, sub-dermal, karatinocytes, melanocytes), trachea, bronchus, lung, heart, umbilical cord, cervix, ovary, testes fibroblast, blood). Cell types that can be utilized herein include fibroblast, epithelial and hematopoietic cells for example. Mammalian cells include without limitation rodent (e.g., mouse, rat, rabbit, hamster), canine, feline, monkey, ape and human cells. Mammalian cells that can be utilized include primary cells derived from a tissue sample, diploid cell strains, transformed cells or established cell lines (e.g., HeLa), each of which may optionally be diseased or genetically altered. Mammalian cells, such as hybridomas, CHO cells, COS cells, VERO cells, HeLa cells, 293 cells, PER-C6 cells, K562 cells, MOLT-4 cells, M1 cells, NS-1 cells, COS-7 cells, MDBK cells, MDCK cells, MRC-5 cells, WI-38 cells, SP2/0 cells, BHK cells (including BHK-21 cells) and derivatives thereof also may be used herein. Insect cells particularly suitable for use in forming such compositions include those derived from Spodoptera species (e.g., Sf9 or Sf21, derived from Spodoptera frugiperda) or Trichoplusa species (e.g., HIGH FIVE™ or MG1, derived from Trichoplusa ni). Cells from cell lines or from primary sources can be useful for certain embodiments. Tissues, organs, organ systems and organisms derived from animals or constructed in vitro or in vivo using methods routine in the art may similarly be used. Cells may be utilized herein in a variety of medical (including diagnostic and therapeutic), industrial, forensic and research applications requiring ready-to-use cultures of animal cells in serum-free media.

Animal cells for culturing and use in conjunction with embodiments of the present invention may be obtained commercially, for example from ATCC (Rockville, Md.), Cell Systems, Inc. (Kirkland, Wash.), Clonetics Corporation (San Diego, Calif.), BioWhittaker (Walkersville, Md.), or Cascade Biologicals (Portland, Oreg.). Alternatively, cells may be isolated directly from samples of animal tissue obtained via biopsy, autopsy, donation or other surgical or medical procedure. Names of cells available from such commercial sources are incorporated by reference herein.

Cells may be derived from tissue in certain embodiments. Tissue generally is handled using standard sterile technique and a laminar flow safety cabinet. In the use and processing of all human tissue, the recommendations of the U.S. Department of Health and Human Services/Centers for Disease Control and Prevention should be followed (Biosafety in Microbiological and Biomedical Laboratories, Richmond, J. Y. et al., Eds., U.S. Government Printing Office, Washington, D.C. 3rd Edition (1993)). The tissue often is cut into small pieces (e.g., 0.5.times.0.5 cm) using sterile surgical instruments. The small pieces generally are washed twice with sterile saline solution supplemented with antibiotics as above, and then may be optionally treated with an enzymatic solution (e.g., collagenase or trypsin solutions, each available commercially, for example, from GIBCO/LTI, Gaithersburg, Md.) to promote dissociation of cells from the tissue matrix.

The mixture of dissociated cells and matrix molecules are washed twice with a suitable physiological saline or tissue culture medium (e.g., Dulbecco's Phosphate Buffered Saline without calcium and magnesium). Between washes, the cells are centrifuged (e.g., at 200.times.g) and then resuspended in serum-free tissue culture medium. Aliquots are counted using an electronic cell counter (such as a Coulter Counter). Alternatively, the cells can be counted manually using a hemocytometer.

The isolated cells can be plated according to the experimental conditions determined by the investigator. Optimal plating and culture conditions for a given animal cell type can be determined by one of ordinary skill in the art using only routine experimentation. For routine culture conditions, using the present invention, cells can be plated onto the surface of culture vessels without attachment factors. Alternatively, the vessels can be pre-coated or contacted with natural, recombinant or synthetic attachment factors or peptide fragments (e.g., collagen or fibronectin, or natural or synthetic fragments thereof). Isolated cells can also be seeded into or onto a natural or synthetic three-dimensional support matrix such as a preformed collagen gel or a synthetic biopolymeric material. Use of attachment factors or a support matrix with the medium of the present invention will enhance cultivation of many attachment-dependent cells in the absence of serum supplementation. Cell seeding densities for each experimental condition may be optimized for the specific culture conditions being used. For routine culture in plastic culture vessels, an initial seeding density of 1-5.times.10.sup.6 cells per cm.sup.2 is preferable.

Mammalian cells typically are cultivated in a cell incubator at about 37.degree. C. The incubator atmosphere should be humidified and should contain about 3-10% carbon dioxide in air, although cultivation of certain cell lines may require as much as 20% carbon dioxide in air for optimal results. Culture medium pH often is in the range of about 7.1-7.6, preferably about 7.1-7.4, and most preferably about 7.1-7.3. Cells in closed or batch culture may undergo complete medium exchange (i.e., replacing spent media with fresh media) about every 1-2 days, or more or less frequently as required by the specific cell type. Cells in perfusion culture (e.g., in bioreactors or fermenters) will receive fresh media on a continuously recirculating basis.

FIG. 14 shows an enlarged view of the cell culture layer 70 disposed on a support layer 72. The cell culture layer 70 includes human stem cells and the support layer 72 includes mouse fibroblast cells, however, any of the cell types, support materials or methods of preparing these described herein may also be useful for some embodiments of the methods and devices discussed herein. The thickness of the cell culture layer 70 disposed on the support layer 72 may be about 5 microns to about 50 microns, more specifically, about 10 microns to about 15 microns, for some cell culture layer embodiments. The cell culture dish 74 may have a diameter of about 10 mm to about 200 mm, more specifically, about 20 mm to about 100 mm, and even more specifically, about 30 mm to about 75 mm and may have a depth of about 3 mm to about 20 mm, more specifically, about 5 mm to about 15 mm for some embodiments.

In order to passage or otherwise cultivate a cell culture and produce a large supply of cells within a particular cell line or having a particular attribute, cells are generally seeded onto a clean sterile support substrate or layer 72 and allowed to proliferate in an environment which is conducive to cell growth. The seeding of cells may be carried out with a particular number of cells or particular cell densities being seeded or otherwise transferred and may be transferred from existing cultures or from primary sources. Cell counts of about 100 cells to about 100,000,000 cells may be transferred or otherwise plated for some embodiments, and, depending on the area occupied by the cells being transferred, which may be in confluence, semi-confluence or non-confluent. Cell densities and determination of confluence may be carried out by any suitable method such as the use of a reticle in the field of view of a microscope, a hemocytometer or the like. With regard to cell passaging, the term as used herein in a general sense is meant to encompass the separation of cells from other cells and exposing the separated cells to new conditions, which may include seeding the separated cells onto a new substrate.

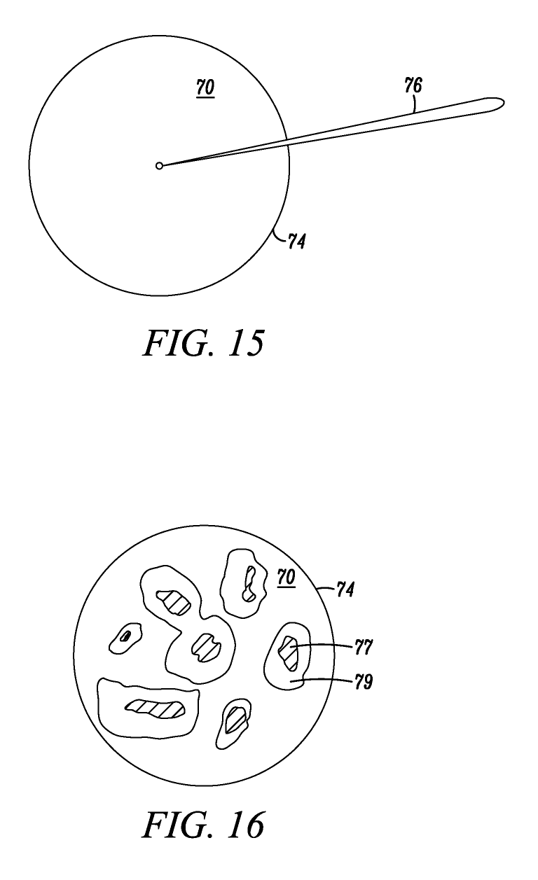

FIG. 15 illustrates a support layer 72 disposed within a cell culture dish 74 being seeded with a pure cell culture line. The seeding is accomplished by placing a number of pure cells from the cell culture on the tip of an elongate fine tipped stylet 76 or other suitable tool and transferring these cells from the tip of the stylet to the support layer 72. The support layer 72 may also be seeded by the application of a liquid solution containing the cells of a desired cell culture to the surface of the support layer 72. FIG. 16 illustrates the cell culture layer 70 in the cell culture dish 74 of FIG. 15 after the cell culture line has proliferated after several heating and cooling cycles.

The cell line shown includes an exemplary human stem cell line which has spread across the top surface of the support layer 72 and has produced zones of differentiated cells 77 and undifferentiated cells 79. For stem cells, the cells tend to stay together or in proximity to adjacent cells, but may begin to differentiate into specific cell types over time, and particularly if the cells become too crowded as a result of cell proliferation. For many applications, it may be desirable to cultivate only the undifferentiated cells and so only those will be transferred to a new cell culture dish 74 for further proliferation. It is the ability to partition a cell culture layer 70 as shown in FIG. 16 into small isolated divided portions that allows a user to select, transfer and further cultivate only those portions of the cell culture layer 70 that are desired.

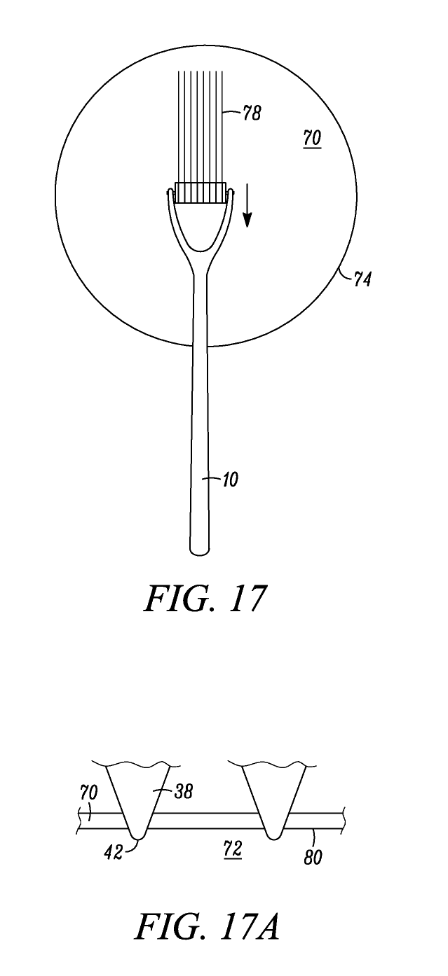

FIG. 17 illustrates the initiation of a first pass of a layer penetrating structure 16 of a roller body 12 of cell processing tool 10 across a cell culture layer 70 disposed in the cell culture dish 74. For such a pass, the roller body 12 is first brought vertically down into the cell culture dish 74 with the roller body 12 against the wall of the cell culture dish 74 with the handle 22 facing away from the wall of the cell culture dish 74. The roller body 12 is brought down into the cell culture dish 74 until the layer penetrating structure 16 makes contact with the cell culture layer 70 and the layer penetrating edges 42 of the circumferential ridges 38 of the layer penetrating structure 16 penetrate and begin to partition the cell culture layer 70 as shown in FIG. 17A. The handle 22 may then be pulled across the cell culture dish 74 while maintaining an appropriate and steady downward pressure on the roller body 12 against the cell culture layer 70 with the layer penetrating structure 16 of the roller body 12 cutting through and partitioning the cell culture layer 70 as the roller body 12 rolls across the cell culture layer 70. This motion creates multiple linear partition lines 78 that are parallel to each other and extend to a depth that is at or below the top surface 80 of the support layer 72 so as to create a full partitioning of the cell culture layer 70 as shown in FIG. 17A.

As discussed above, during such a pass of the roller body 12, it may be desirable for the layer penetrating structure 16 to roll over and partition the cell culture layer 70 without sliding or skidding on the cell culture layer 70 or the support substrate 72 in order to avoid cross contamination or deformation of either of the layers. In addition, it may be desirable for the layer penetrating structure 16 to contact the cell culture layer 70 and be pulled away from the cell culture layer 70 during the partitioning process without picking up cells or otherwise having cells of the cell culture layer 70 adhere to the layer penetrating structure 16. This may be achieved in some embodiments by having a cell culture layer 70 with particular surface properties and a layer penetrating structure 16 with an outer surface that is smooth and non-adherent.

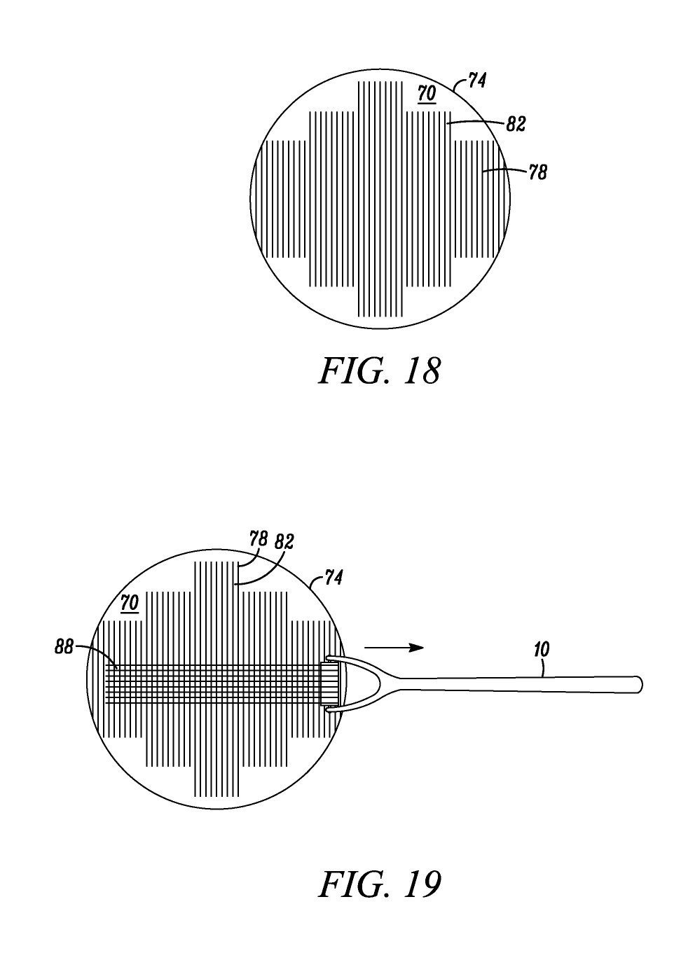

Once the first pass is completed, the process may be repeated as many times as necessary by repositioning the roller body 12 adjacent to the partition lines 78 of the first pass and passing the roller body 12 across the cell culture layer 70 adjacent and parallel the first pass to produce a second set of partition lines 78 adjacent and parallel the first. The process may be repeated until the entire surface, or most of the surface, of the cell culture layer 70 has been partitioned into elongate parallel strips 82 with the partitions 78 extending at least from a top surface 84 of the cell culture layer 70 to a bottom surface of the cell culture layer 70 as shown in FIG. 18. Because of its cylindrical configuration, the roller body 86 is not able to make contact with the cell culture layer 70 directly against the wall of the cell culture dish 74 and the partition lines 78 in the cell culture layer 70 begin at a position that is approximately one the distance of one radius of the roller body 12 from the wall of the cell culture dish 74. As such, it should be noted that the partitioned strips 78 of the cell culture layer 70 shown in FIG. 18 are not isolated divided portions as the strips 82 are connected to adjacent strips 82 at their ends next to the wall of the cell culture dish 74.

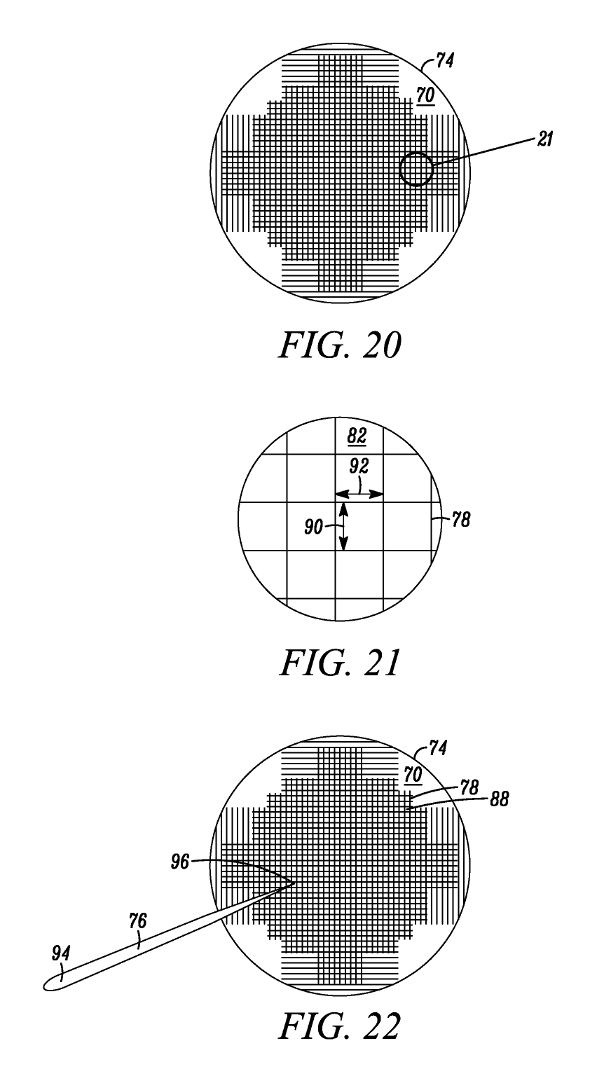

Once this set of partitions 78 has been produced, a second set of partition lines 78 or furrows, which may be substantially perpendicular to the first set of partition lines 78 for some embodiments, are produced in the same way as described above with the same cell culture layer processing tool 10 as shown in FIG. 19. As the layer penetrating structure 16 of the roller body 12 is drawn across the cell culture layer 70 in a second direction that is different from the direction of the first set of partition lines 78, the cell culture layer 70 is then partitioned into isolated divided portions 88 that are completely partitioned from adjacent portions of the cell culture layer 70. The result is shown in FIG. 20 wherein the majority of the cell culture layer 70 has been partitioned into isolated square shaped divided portions 88 having a relatively small surface area with partition cuts 78 extending through the entire cell culture layer 70 about the entire boundary of each divided portion 88. Note that some divided portions 88 may not have this desired attribute and may not be fully partitioned, particularly at the edges or boundaries of the partitioned portion of the cell culture layer 70 adjacent the wall of the cell culture dish 74. For some embodiments, the divided portions 88 may be substantially square sections having sides with a length, as indicated by arrows 90 and 92 in FIG. 21, of about 50 microns to about 1000 microns, more specifically, about 100 microns to about 500 microns, and even more specifically, about 150 microns to about 250 microns. The cell culture processing tool may also be used to create diamond shaped isolated divided portions of a cell culture layer, as well as other shapes, if the layer penetrating structure 16 is advanced across the cell culture layer in different directions that are not substantially perpendicular to each other or are performed in a curved path.

Once the cell culture layer 70 or a desired portion thereof has been partitioned into isolated divided portions 88, each isolated divided portion having substantially the same surface area dimensions, the divided portions 88 may then be removed from the cell culture dish 74 and support layer 72 for any desired purpose by a variety of methods. The isolated divided portions 88 of the cell culture layer to be removed may be selected for any desired attribute or characteristic, such as any of the attributes or characteristics of cells discussed above. For example, it may be desirable to remove and transport all isolated divided portions 88 of the cell culture layer having cells in a desired state, such as a differentiated state or undifferentiated state. FIG. 22 illustrates an isolated divided portion 88 of the cell culture 70 layer being removed by an elongate fine tipped stylet 76 for transport to another location. The stylet 76 shown has a handle portion 94 and a fine tip point 96, which may be barbed for some embodiments, suitable for lifting the isolated divided portions 88 of the cell culture layer 70 from the support layer 72. As such, the fine tip point 96 may be a sharpened tip, a flattened tip, a hollow tip, barbed tip or any other suitable configuration that would facilitate the lifting and holding of a divided portion 88 of the cell culture layer 70. Once the divided portion 88 of the cell culture layer 70 has been removed from the cell culture dish 74, it may then be transported and used to seed another new cell culture dish 74 as shown in FIG. 15 and discussed above for further passaging.

The divided portion 88 may also be transferred to a tube (not shown) or other suitable vessel for spin down as well as other processing. For example, in some embodiments, the divided portion 88 may be transferred to a solution in a 15 ml tube for spin down at about 800 to about 1200 rpm for about 1.5 minutes to about 2.5 minutes. The solution in the tube may also be left to stand stationary over a period of time suitable for the cells in the solution to settle to the bottom of the tube by gravity. The cells may then be returned singly and re-suspended in a new growth medium and transferred to a new support layer. Once transferred and seeded, the newly seeded cell culture dish may be allowed to proliferate with at least one heating and cooling cycle or other environmental support that is conducive to cell growth and multiplication. For some embodiments, multiple heating and cooling cycles may be used to promote cell growth. In this way, the desirable features of the initial cell line used to seed the initial cell culture dish 74 may be cultivated and expanded to produce a high volume of cells from the line with desirable traits. It may also be desirable to perform some or all of the procedures discussed above under a laminar flow hood or in another type of suitable purified environment to avoid contamination of the cell culture.

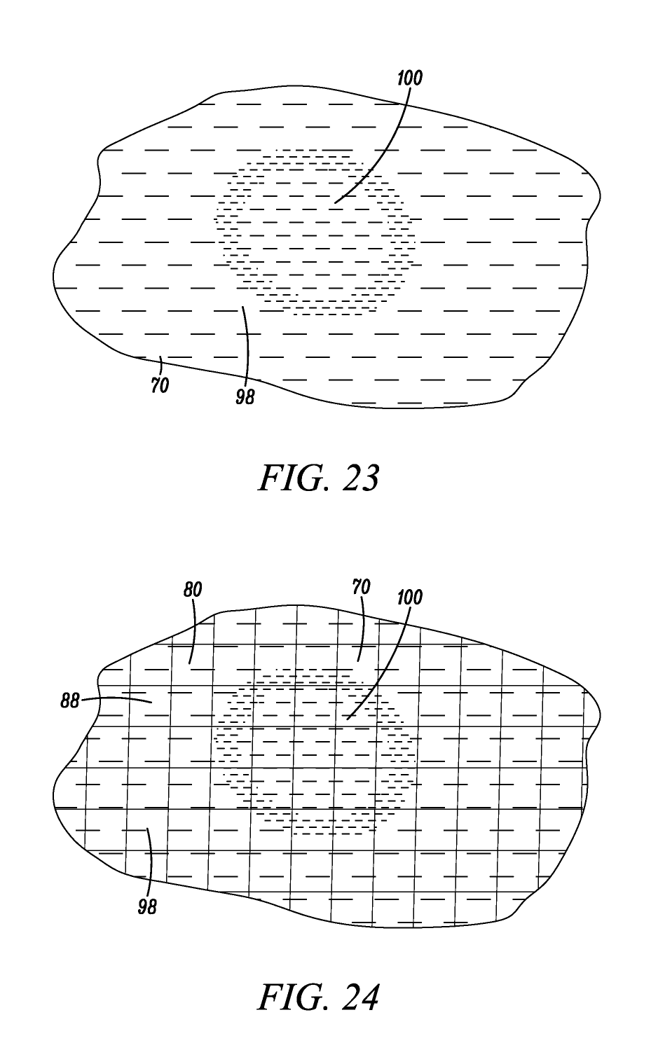

A schematic illustration of a cell culture layer 70 of human stem cells is shown in FIG. 23. Similarly configured cell culture layers may include other types of cells as well, including non-human stem cells, embryoid bodies, neurospheres and the like, as well as any of the other cell types discussed above. The cell culture layer 70 includes a portion of cells 98 in an undifferentiated state and a portion of cells 100 in a differentiated state. For some cell culturing methods, it is desirable to divide the cell culture layer 70 that has undifferentiated cells into isolated divided portions 88 having substantially the same surface area and substantially the same number of undifferentiated cells in each divided portion 88. These divided portions 88 may then be transferred to respective cell culture dishes so as to seed the cell culture dishes 74 with approximately the same number of the same type of cells which may be useful for the creation and processing of large volumes of desirable cells. For other embodiments, it may be desirable to transport and further cultivate divided portions 88 that have cells which are in a differentiated state. FIG. 24 shows the cell culture layer 70 of FIG. 23 which has been partitioned into isolated divided portions 88. The isolated divided portions 88 are generally square in shape and may have sides with a length of about 220 microns to about 240 microns. Such isolated divided portions 88 may be transferred to another cell culture dish 74 or for any other desired purpose.

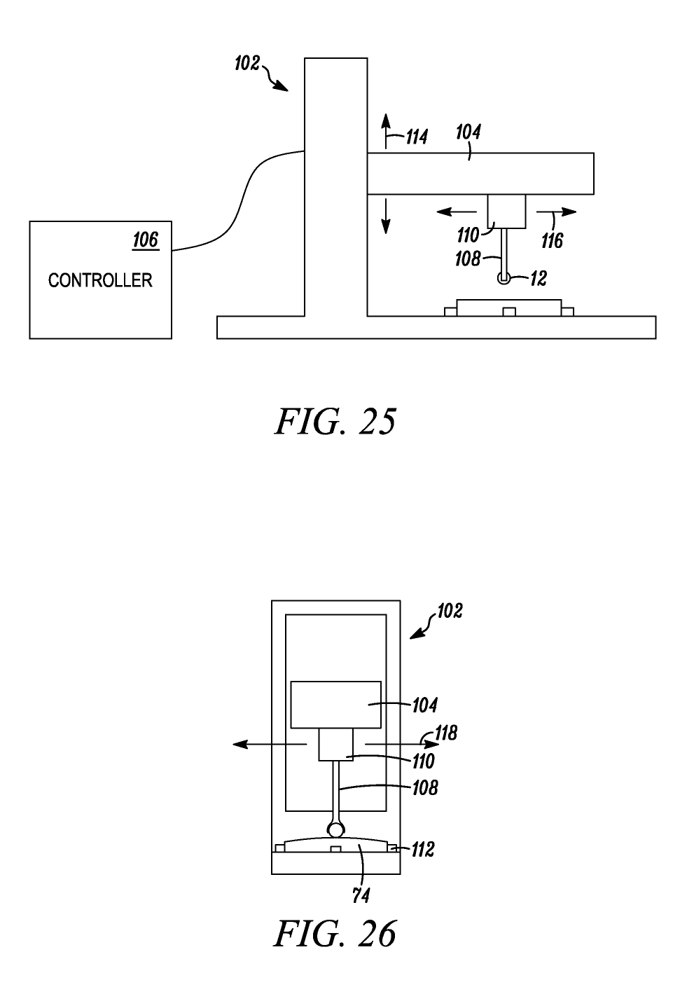

Although the cell layer processing tool embodiments described above have been primarily directed to a hand operated configurations, it may be desirable for some applications to use the same or similar roller body 12 embodiments in an automated format. FIGS. 25 and 26 illustrate an embodiment of a robotic cell culture processing tool 102. Embodiments of the robotic cell culture processing tool 102 may include a three axis robotic positioning actuator 104, a controller 106 coupled to the three axis robotic positioning actuator 104 and a roller body 12 having a substantially cylindrical non-adherent outer surface 14 with a layer penetrating structure 16 configured to penetrate a cell culture layer 70 into divided portions disposed thereon.

A support structure 108 is coupled to the roller body 12 and is configured to allow rotation of the roller body 12 about an axis that is substantially concentric with the cylindrical outer surface 14 of the roller body 12. The support structure 108 is secured to a movable carrier 110 of the three axis robotic positioning actuator 104. With this arrangement, the controller 106 may include a processor (not shown) that may be programmed to move the moveable carrier 110 in a controllable manner in each of three orthogonal axes so as to controllably position and move the roller body 12. In this way, the roller body 12 and layer penetrating structure 16 thereof may be positioned and moved across a cell culture layer 70 in the same way as the roller body embodiments discussed above. This allows a cell culture layer 70 to be partitioned into divided portions 88 as discussed above in a repeatable manner without the need for a human operator and thus reducing human/operator error.

A cell culture dish 74 having a cell culture layer 70 disposed on a support layer 72 in the cell culture dish 74 may be placed under the roller body 12 which is coupled to the moveable carrier 110 by the support structure 108. The cell culture dish 74 may be fixed in the x-y plane position by a plurality of stops 112 that are configured to hold the cell culture dish 74 in place by spacing them around a circular radius that is just slightly larger than an outer radius of the pre-selected cell culture dish 74. The movable carrier 110 and roller body 12 coupled thereto may then be lowered into contact with the cell culture layer 70 and moved across the cell culture layer 70 with a substantially constant downward force to partition the cell culture layer 70 into divided portions 88. The moveable carrier 110 may be actuated by a servo motor or the like corresponding to each axis of the three axis positioning actuator 104. There may be a servo motor or the like configured to move the moveable carrier 110 in a z-axis direction as indicated by arrows 114, in the y-axis direction as indicated by arrows 116 and in the x-axis direction as indicated by arrows 118.

For some embodiments of cell culture processing tools, a method of use may include specific procedures in order to obtain desired results. The following discussion includes a series of exemplary procedures for use of a cell culture processing tool, such as cell culture processing tool 10 or any of the other cell culture processing tools, or components thereof, discussed above. For some such embodiments, the processing tool represents a novel human embryonic stem cell passaging device that makes manual passaging of stem cell colonies more rapid and reproducible. The processing tool is made of a cell-culture safe inert material that facilitates cutting of the embryonic stem cell colonies growing on feeder cells or other substrate into uniform sized pieces for reproducible and optimal passaging. This tool outperforms existing manual and enzymatic passing methods in speed, uniformity of passaged colonies, and reliability and should be stored at room temperature.

Some advantages of the cell culture processing tool may include manual passaging stem cell colonies in a fraction of the time compared with standard techniques, stem cell colonies are cut in pieces of uniform size, making passaging of stem cells more reproducible and the processing tool is cell-culture safe, ready for-use and packaged individually in gamma-irradiated sleeves. During use, procedures often are performed aseptically under a laminar flow hood. This embodiment of the cell culture processing tool is intended for one-time use and users generally do not sterilize (alcohol or autoclave) the processing tool, since the tool may lose shape and cease to function properly.

In use, the differentiated portions of human embryonic stem cell culture may optionally be dissected out using a poker or spike and thereafter removed by changing the medium. In general, the processing tool is used by first pulling open packaging and removing the processing tool 10 under a laminar flow hood. A culture vessel is held in one hand and the processing tool pulled across the entire plate in one direction as shown in FIG. 17 discussed above. Enough pressure should be applied so the entire roller blade 12 touches the plate and uniform pressure is maintained during the rolling action. The culture medium generally is not removed before rolling the plate. The processing tool is pulled or advanced again parallel to the first pass until the entire plate has been covered. Thereafter, the culture vessel is rotated 90 degrees, and the rolling steps repeated as shown in FIG. 19 discussed above. Using a serological pipette, the plate is rinsed using the medium on the plate so that the cut colonies are suspended in the medium. The medium containing colonies may be transferred to a 15 ml tube and spun down at 1000 rpm (200 g) for 2 min. Alternatively, the tube may be left in a stand so that the colonies settle by gravity. The supernatant is aspirated carefully to remove single cells or contaminating feeder cells (MEFs) from the population. The colonies are then re-suspended in medium and transfer to the new matrix (typically at a 1:4 passaging rate). The processing tool is then discarded after use with no re-use. A typical example of a human embryonic stem cell culture plate before and after cutting the colony with the processing tool 10 is shown, as seen under 4× magnification in phase microscopy, is depicted in FIGS. 23 and 24, respectively. FIG. 23 shows the cell culture plate with colony before cutting. FIG. 24 shows the cell culture plate after cutting colony.

With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments of the invention. For example, any of the roller body embodiments 12, 48 and 54 may be used in conjunction with handle 22 or the robotic cell culture processing tool 102. Accordingly, it is not intended that the invention be limited by the forgoing detailed description.

What is claimed is:

1. A cell culture processing device, comprising:
a roller body having a substantially cylindrical non-adherent outer surface with a layer penetrating structure disposed thereon and configured to penetrate a cell culture layer into divided portions; and
a support structure coupled to the roller body and configured to allow rotation of the roller body about an axis that is substantially concentric with the cylindrical outer surface.

2. The processing device of claim 1 wherein the layer penetrating structure comprises a plurality of adjacent circumferential ridges regularly spaced in an axial direction.

3. The processing device of claim 2 wherein the plurality of adjacent circumferential ridges comprise a single helical ridge extending from a first portion of the roller body to a second portion of the roller body which is axially spaced from the first portion.

4. The processing device of claim 2 wherein the circumferential ridges are about 0.005 inches to about 0.015 inches high.

5. The processing device of claim 2 wherein the circumferential ridges are spaced about 0.005 inches to about 0.015 inches apart.

6. The processing device of claim 1 wherein the layer penetrating structure comprises ridges surrounding substantially closed boundaries disposed at regularly space intervals on the cylindrical outer surface.

7. The processing device of claim 6 wherein the substantially closed boundaries have a rectangular shape.

8. The processing device of claim 1 wherein the outer surface of the roller body comprises an elastomeric material.

9. The processing device of claim 8 wherein the elastomeric material comprises silicone rubber.

10. The processing device of claim 9 wherein the silicone rubber comprises a shore hardness of about 65 A shore hardness to about 75 A shore hardness.

11. The processing device of claim 1 wherein the support structure comprises an axle extending coaxially through the roller body.

12. The processing device of claim 11 wherein the support structure further comprises a handle coupled to the axle so as to allow relative rotational movement between the handle and the axle.

13. A cell culture processing tool, comprising:
a roller body having a substantially cylindrical non-adherent outer surface with a layer penetrating structure configured to penetrate a cell culture layer into divided portions disposed thereon;
an axle extending coaxially through the roller body and configured to support smooth rotational movement of the roller body about a longitudinal axis of the roller body; and
a handle coupled to the axle.

14. The processing tool of claim 13 wherein the layer penetrating structure comprises a plurality of adjacent circumferential ridges regularly spaced in an axial direction.

15. The processing tool of claim 14 wherein the plurality of adjacent circumferential ridges comprise a single helical ridge extending from a first portion of the roller body to a second portion of the roller body which is axially spaced from the first portion.

16. The processing tool of claim 14 wherein the circumferential ridges are about 0.005 inches to about 0.015 inches high.

17. The processing tool of claim 14 wherein the circumferential ridges are spaced about 0.005 inches to about 0.015 inches apart.

18. The processing tool of claim 13 wherein the layer penetrating structure comprises ridges surrounding substantially closed boundaries disposed at regularly space intervals on the cylindrical outer surface.

19. The processing tool of claim 18 wherein the substantially closed boundaries are rectangular shaped.

20. The processing tool of claim 13 wherein the outer surface of the roller body comprises an elastomeric material.

21. The processing tool of claim 20 wherein the elastomeric material comprises silicone rubber.

22. The processing tool of claim 21 wherein the silicone rubber comprises a shore hardness of about 65 A shore hardness to about 75 A shore hardness.

23. The processing tool of claim 13 wherein the handle comprises an elongate handle body with a deflected distal section that forms an angle with a nominal longitudinal axis of the elongate handle body and the axle is coupled to the deflected distal section.

24. The processing tool of claim 23 the deflected distal section of the handle body has a length of about 1.0 inch to about 1.5 inches and forms an angle of about 120 degrees to about 150 degrees with respect to the nominal longitudinal axis of the elongate handle body.

25. The processing tool of claim 23 wherein the deflected distal section has a bifurcated structure with distal portions having opposed recesses that capture the respective ends of the axle and allow rotation of the axle within the recesses.

26. The processing tool of claim 25 wherein the longitudinal axis of the axle is substantially perpendicular to a plane defined by a longitudinal axis of the deflected distal section and a longitudinal axis of the elongate handle body proximal of the deflected distal section.

27. A robotic cell culture processing tool comprising
a three axis robotic positioning actuator;
a controller coupled to the three axis robotic positioning actuator;
a roller body having a substantially cylindrical non-adherent outer surface with a layer penetrating structure configured to penetrate a cell culture layer into divided portions disposed thereon; and
a support structure which is coupled to the roller body configured to allow rotation of the roller body about an axis that is substantially concentric with the cylindrical outer surface and which is secured to a movable carrier of the three axis robotic positioning actuator.

28. The processing tool of claim 27 wherein the layer penetrating structure comprises a plurality of adjacent circumferential ridges regularly spaced in an axial direction.

29. The processing tool of claim 28 wherein the plurality of adjacent circumferential ridges comprise a single helical ridge extending from a first portion of the roller body to a second portion of the roller body which is axially spaced from the first portion.

30. The processing tool of claim 28 wherein the circumferential ridges are about 0.005 inches to about 0.015 inches high.

31. The processing tool of claim 28 wherein the circumferential ridges are spaced about 0.005 inches to about 0.015 inches apart.

32. The processing tool of claim 27 wherein the layer penetrating structure comprises ridges surrounding substantially closed boundaries disposed at regularly space intervals on the cylindrical outer surface.

33. The processing tool of claim 32 wherein the substantially closed boundaries are rectangular shaped.

34. The processing tool of claim 27 wherein the outer surface of the roller body comprises an elastomeric material.

35. The processing tool of claim 34 wherein the elastomeric material comprises silicone rubber.

36. The processing tool of claim 35 wherein the silicone rubber comprises a shore hardness of about 65 A shore hardness to about 75 A shore hardness.

37. The processing tool of claim 27 wherein the three axis robotic positioning actuator comprises a servo motor corresponding to each axis and configured to actuate the moveable carrier in a direction corresponding to each axis.

38. The processing tool of claim 27 wherein the controller comprises a processor which is programmed to controllably move the roller body in a pre-determined pattern of motion.

39. The processing tool of claim 38 wherein the pre-determined pattern of motion comprises at least one linear pass across a cell culture layer disposed in a cell culture dish.

40. A method of processing a cell culture layer comprising partitioning the cell culture layer by advancing a roller body having a layer penetrating raised structure across the cell culture layer while applying a predetermined amount of force against the cell culture layer to cut through the cell culture layer and separate the cell culture layer into divided portions.

41. The method of claim 40 wherein the layer penetrating structure comprises ridges surrounding substantially closed boundaries disposed at regularly spaced intervals on a cylindrical outer surface of the roller body and a single pass of the roller body across the cell culture layer is used to separate cell culture layer into isolated divided portions.

42. The method of claim 40 wherein the layer penetrating structure of the roller body comprises circumferential ridges which are axially spaced from each other and at least two passes of roller body across the cell culture layer in at least two different directions are used to separate the cell culture layer into isolated divided portions.

43. A method of processing a cell culture, comprising:
seeding a cell culture support layer with cells;
allowing the cells to proliferate on the cell culture support layer and form a cell culture layer disposed on a cell culture support layer top surface; and
partitioning the cell culture layer by advancing a roller body having a layer penetrating structure across the cell culture layer while applying a predetermined amount of force against the cell culture layer to cut through the cell culture layer and separate the cell culture layer into divided portions.

44. The method of claim 43 wherein partitioning the cell culture layer comprises partitioning the cell culture layer into isolated divided portions with a single pass of a roller body having a layer penetrating structure which comprises ridges surrounding substantially closed boundaries disposed at regularly space intervals on a cylindrical outer surface of the roller body.

45. The method of claim 43 wherein partitioning the cell culture layer comprises partitioning the cell culture layer into isolated divided portions with two passes of the roller body across the cell culture layer in different directions.

46. The method of claim 45 wherein the layer penetrating structure comprises a plurality of adjacent circumferential ridges regularly spaced in an axial direction on a cylindrical outer surface of the roller body and advancing the roller body across the cell culture layer produces divided portions in the configuration of elongated strips of cell culture layer.

47. The method of claim 43 wherein seeding a cell culture support substrate with a cell line comprises seeding a cell culture support substrate with a human stem cell line.

48. The method of claim 43 wherein allowing the cells of the cell line to proliferate on the cell culture substrate and form a cell culture layer disposed on the cell culture substrate surface comprises imposing at least one heating and cooling cycle on the cell culture layer after seeding.

49. A method for passaging cells, comprising
partitioning a cell culture layer with a cell culture processing tool by rolling a layer penetrating structure of the cell culture processing tool over the cell culture layer so as to partition the layer of cells into a plurality of isolated divided portions;
selecting an isolated divided portion of the layer of cells having cells with at least one predetermined characteristic;
transporting the isolated divided portion to a new location; and
allowing the transported cells to proliferate in the new location and generate a new cell culture layer of cells having the predetermined characteristic.

50. The method of claim 49 wherein partitioning the cell culture layer comprises partitioning the cell culture layer into isolated divided portions with a single pass of a roller body having a layer penetrating structure which comprises ridges surrounding substantially closed boundaries disposed at regularly space intervals on a cylindrical outer surface of the roller body.

51. The method of claim 49 wherein the layer penetrating structure comprises a plurality of adjacent circumferential ridges regularly spaced in an axial direction on a cylindrical outer surface of the roller body and wherein partitioning the cell culture layer comprises partitioning the cell culture layer into isolated divided portions with two passes of the roller body across the cell culture layer in different directions.

52. The method of claim 49 further comprising repeating the method for about 1 passage to about 1000 passages.

53. A method of maintaining a cell line in a desired state, comprising
partitioning a cell culture layer with a cell culture processing tool by rolling a layer penetrating structure of the cell culture processing tool over the cell culture layer so as to partition the layer of cells into a plurality of isolated divided portions;
selecting an isolated divided portion of the layer of cells having cells in a predetermined state;
transporting the isolated divided portion to a new location; and
allowing the transported cells to proliferate in the new location and generate a new cell culture layer of cells having the predetermined state.

54. The method of claim 53 wherein the predetermined state comprises a differentiated state.

55. The method of claim 53 wherein the predetermined state comprises an undifferentiated state.

56. The method of claim 53 wherein partitioning the cell culture layer comprises partitioning the cell culture layer into isolated divided portions with a single pass of a roller body having a layer penetrating structure which comprises ridges surrounding substantially closed boundaries disposed at regularly space intervals on a cylindrical outer surface of the roller body.

57. The method of claim 53 wherein the layer penetrating structure comprises a plurality of adjacent circumferential ridges regularly spaced in an axial direction on a cylindrical outer surface of the roller body and wherein partitioning the cell culture layer comprises partitioning the cell culture layer into isolated divided portions with two passes of the roller body across the cell culture layer in different directions.
